(19) United States
(12) Patent Application Publication
(10) Pub. No.: US 2025/0069731 A1
(43) Pub. Date: Feb. 27, 2025

(54) PROCESSING OF MEDICAL DIAGNOSTIC DATA TO OBTAIN CODING INFORMATION

(71) Applicant: Royal Bank of Canada, Toronto (CA)

(72) Inventors: Avideh KHALILI, Thornhill (CA); Zachary James FONG, Burnaby (CA); Justtin DaMinh HOANG, Mississauga (CA); Ramin KAHIDI, Calgary (CA); Kevin McLOUGHLIN, Toronto (CA); Linda KALEIS, Aurora (CA); Laura HALLIDAY, Collingwood (CA); Marco CIRILLO, Toronto (CA); Alexander CHAN, Markham (CA); Richard LANGLOIS, Amherstview (CA)

(21) Appl. No.: 18/799,816

(22) Filed: Aug. 9, 2024

Related U.S. Application Data
(60) Provisional application No. 63/534,215, filed on Aug. 23, 2023.

(51) Int. Cl. G16H 40/20; G06Q 30/04

(52) U.S. Cl. CPC G16H 40/20; G06Q 30/04

(57) ABSTRACT

Processing of medical diagnostic information returns comprises receiving a text block containing medical diagnostic information and returning corresponding tracking codes. Diagnostic terms may be identified within a text block containing medical diagnostic information, and the diagnostic terms may be mapped to corresponding respective tracking codes. At least one trained machine learning model, such as a large language model and/or a classifier, may be used to identify the tracking codes that correspond to the diagnostic terms.

### CROSS-REFERENCE TO RELATED APPLICATION

This application claims the benefit of U.S. Provisional Application No. 63/534,215 filed on Aug. 23, 2023, the teachings of which are hereby incorporated by reference.

### TECHNICAL FIELD

The present disclosure is directed to processing of medical diagnostic data, and more particularly to processing of medical diagnostic data to obtain coding information.

### BACKGROUND

In many jurisdictions, physicians are in short supply. For example, the report entitled The Complexities of Physician Supply and Demand: Projections From 2019 to 2034 prepared for the Association of American Medical Colleges and dated June 2021 expects a shortfall of between 37,800 and 124,000 physicians in the United States by 2034. Similarly, in the province of Ontario, Canada, as of March 2022 there were more than two million people without a regular family doctor.

Given these expected shortages, a doctor's time is a critical resource that must be used judiciously. Every minute spent by a doctor on administrative tasks is time that cannot be spent with a patient. Yet, medical billing is complex and intensive for physicians, squandering time that can never be recovered (and possibly impacting the actual recovery of real people). For example, many physicians use a fee-for-service model, where they operate as a self-employed professional. Therefore, they need to submit billing claims to a third party payor, such as an insurer or a government agency, in order to be compensated. Creating these claims involves entering specific codes for each patient, such as service codes and diagnostic codes, onto a separate claim form. Even where the forms can be generated and submitted electronically, just figuring out which codes to use is a daunting task. For example, in Ontario there are over 7000 unique billing and diagnostic codes for physicians to navigate which is often challenging due to the complex interactions and requirements related to the different codes.

The current medical billing solutions on the market require physicians to manually enter codes into their claim forms, offering limited hard-coded rule-based code suggestions. The process of understanding and correctly identifying which service code(s) are relevant to each patient encounter can take hours of a physician's time throughout the week. Formal training on medical billing is generally not provided during medical school or residency. If a physician makes any mistakes in filling out their claim form, their claim may be rejected by the third party payor leading to the physician not being paid for that claim on time if at all. Either of these circumstances can lead to frustration, increasing the chances of burnout that can further reduce the number of available physicians.

Therefore, there is a need for a technical solution to improve the efficiency with which physicians can identify the appropriate service codes and diagnostic codes so that they can spend more time providing critical medical care to the patients who need it most.

### SUMMARY

The present disclosure describes a tool that helps streamline the process of creating support documentation for physicians. Physicians will dictate or type into a computer or mobile device the patient's diagnoses, a description of the service provided, and optionally the time and date of the service. Systems and methods according to the present disclosure can then parse this input into recommendations for the correct service code(s) and diagnostic code(s).

In one aspect, a computer-implemented method for processing medical diagnostic information comprises obtaining diagnostic term information identifying diagnostic terms within a text block containing medical diagnostic information, mapping the identified diagnostic terms to corresponding respective tracking codes to identify matching tracking codes that correspond to the identified diagnostic terms, and returning the matching tracking codes.

The tracking codes preferably comprise at least service codes, and more preferably further comprise diagnostic codes.

The method may further comprise identifying specific instances of the tracking codes within the text block, in which case the returned matching tracking codes include the specific instances of the tracking codes.

In some embodiments, obtaining the diagnostic term information identifying the diagnostic terms within the text block comprises comparing text strings within the text block to a predetermined list of diagnostic terms. In particular embodiments, comparing text strings within the text block to the predetermined list of diagnostic terms comprises comparing text embeddings for the text strings to text embeddings for the predetermined list of diagnostic terms.

In some embodiments, obtaining the diagnostic term information identifying the diagnostic terms within the text block comprises submitting the text block to a trained large language model (LLM) with a prompt for the LLM to return diagnostic term information identifying the diagnostic terms, and receiving the diagnostic term information from the LLM. In some such embodiments, mapping the identified diagnostic terms to corresponding respective tracking codes to identify the matching tracking codes that correspond to the identified diagnostic terms comprises calculating diagnostic term text embeddings for the identified diagnostic terms and comparing the diagnostic term text embeddings to tracking code text embeddings for the tracking codes to identify as candidate tracking codes those of the tracking codes whose respective tracking code text embeddings most closely match the diagnostic term text embeddings. Mapping the identified diagnostic terms to corresponding respective tracking codes to identify the matching tracking codes may further comprise sending highest ranked ones of the candidate tracking codes to the LLM along with the text block and a second prompt for the LLM to return the matching tracking codes, and receiving the matching tracking codes from the LLM.

In another embodiment, obtaining the diagnostic term information identifying the diagnostic terms within the text block comprises submitting the text block to a trained classifier, and receiving diagnostic term information identifying the diagnostic terms from the classifier.

The text block may be a transcript of an audio stream containing spoken words.

In some embodiments, mapping the identified diagnostic terms to the corresponding respective tracking codes comprises calculating diagnostic term text embeddings for the identified diagnostic terms and comparing the diagnostic term text embeddings to tracking code text embeddings for tracking codes to identify those of the tracking codes whose respective tracking code text embeddings most closely match the diagnostic term text embeddings. In such embodiments, the returned matching tracking codes include those of the tracking codes whose respective tracking code text embeddings most closely match the diagnostic term text embeddings. The tracking code text embeddings may be pre-calculated. Comparing the diagnostic term text embeddings to the tracking code text embeddings for the tracking codes may comprise comparison by mathematical distance between vectors. Mapping the identified diagnostic terms to the corresponding respective tracking codes may comprise mapping by semantic similarity.

In some embodiments, mapping the identified diagnostic terms to the corresponding respective tracking codes may comprise comparing the diagnostic terms to a correspondence table in which ones of the diagnostic terms correspond to respective ones of the tracking codes.

In some embodiments, mapping the identified diagnostic terms to the corresponding respective tracking codes comprises providing the diagnostic terms to a trained classifier, and receiving the matching tracking codes from the trained classifier.

In some embodiments, mapping the identified diagnostic terms to the corresponding respective tracking codes comprises providing the diagnostic terms to a trained LLM along with a prompt requesting the matching tracking codes, and receiving the matching tracking codes from the trained LLM.

In another aspect, a method for processing medical diagnostic information comprises receiving a text block containing diagnostic terms, using at least one trained machine learning model to identify matching tracking codes that correspond to the diagnostic terms, and returning the matching tracking codes.

In some embodiments, using at least one trained machine learning model to identify matching tracking codes that correspond to the diagnostic terms comprises submitting the text block to a trained LLM with a first prompt for the LLM to return diagnostic term information identifying the diagnostic terms, and receiving the diagnostic term information from the LLM. In some such embodiments, using at least one trained machine learning model to identify matching tracking codes that correspond to the diagnostic terms further comprises calculating diagnostic term text embeddings for the identified diagnostic terms, and comparing the diagnostic term text embeddings to tracking code text embeddings for the tracking codes to identify as candidate tracking codes those of the tracking codes whose respective tracking code text embeddings most closely match the diagnostic term text embeddings. In particular implementations, using at least one trained machine learning model to identify matching tracking codes that correspond to the diagnostic terms further comprises sending highest ranked ones of the candidate tracking codes to the LLM along with the text block and a second prompt for the LLM to return the matching tracking codes, and receiving the matching tracking codes from the LLM.

In some embodiments, using at least one trained machine learning model to identify matching tracking codes that correspond to the diagnostic terms comprises using an identifier machine learning model to identify diagnostic terms within the text block.

In some embodiments, using at least one trained machine learning model to identify matching tracking codes that correspond to the diagnostic terms comprises using a mapping machine learning model to map the diagnostic terms to corresponding respective tracking codes.

In some embodiments, using at least one trained machine learning model to identify matching tracking codes that correspond to the diagnostic terms comprises using a single trained machine learning model to identify the matching tracking codes that correspond to the diagnostic terms.

The text block may be a transcript of an audio stream containing spoken words received by a microphone of a data processing system.

In other aspects, data processing systems and computer program products for implementing the above-described methods are also provided.

### BRIEF DESCRIPTION OF THE DRAWINGS

In the accompanying drawings, which illustrate one or more example embodiments:

FIG. 1 shows a computer network that comprises an example embodiment of a system for processing medical diagnostic data;

FIG. 2 depicts an example embodiment of a server in a data center;

FIG. 3 shows an overview schematic representation of a method for processing medical diagnostic information according to an aspect of the present disclosure;

FIG. 3A shows an illustrative embodiment of the method of FIG. 3 in which diagnostic terms within a text block are identified by comparing text strings within the text block to a predetermined list of diagnostic terms;

FIG. 3B shows an illustrative embodiment of the method of FIG. 3 in which diagnostic terms within a text block are identified by submitting the text block to a trained large language model (LLM) along with an engineered prompt;

FIG. 3C shows an illustrative embodiment of the method of FIG. 3 in which diagnostic terms within a text block are identified by submitting the text block to a trained classifier;

FIG. 3D shows a schematic representation of a method for processing medical diagnostic information in which an identifier machine learning model is used to identify diagnostic terms within a text block and a mapping machine learning model is used to map the diagnostic terms to corresponding respective tracking codes;

FIG. 3E shows a schematic representation of a method for processing medical diagnostic information in which a single trained machine learning model is used to identify tracking codes that correspond to diagnostic terms within a text block;

FIG. 3F shows a particular implementation of the method shown in FIG. 3E;

FIG. 3G shows an illustrative embodiment of the method of FIG. 3 in which diagnostic terms are mapped to tracking codes using a correspondence table;

FIG. 3H shows an illustrative embodiment of the method of FIG. 3 in which diagnostic terms are mapped to tracking codes using semantic similarity;

FIG. 4 is a flow chart showing a first illustrative method for processing medical diagnostic information according to an aspect of the present disclosure;

FIG. 5 is a flow chart showing a second illustrative method for processing medical diagnostic information according to an aspect of the present disclosure;

FIG. 5A is a flow chart showing an illustrative method of using a trained machine learning model, and in particular an LLM, to identify matching tracking codes that correspond to diagnostic terms; and

FIG. 6 is an illustrative process flow diagram for an illustrative implementation of the method shown in FIG. 5.

### DETAILED DESCRIPTION

Broadly speaking, the present disclosure describes a system, method and computer program product that use the natural language descriptions of the services clinicians have provided for patients to extract the relevant billing and diagnostic codes for that patient encounter.

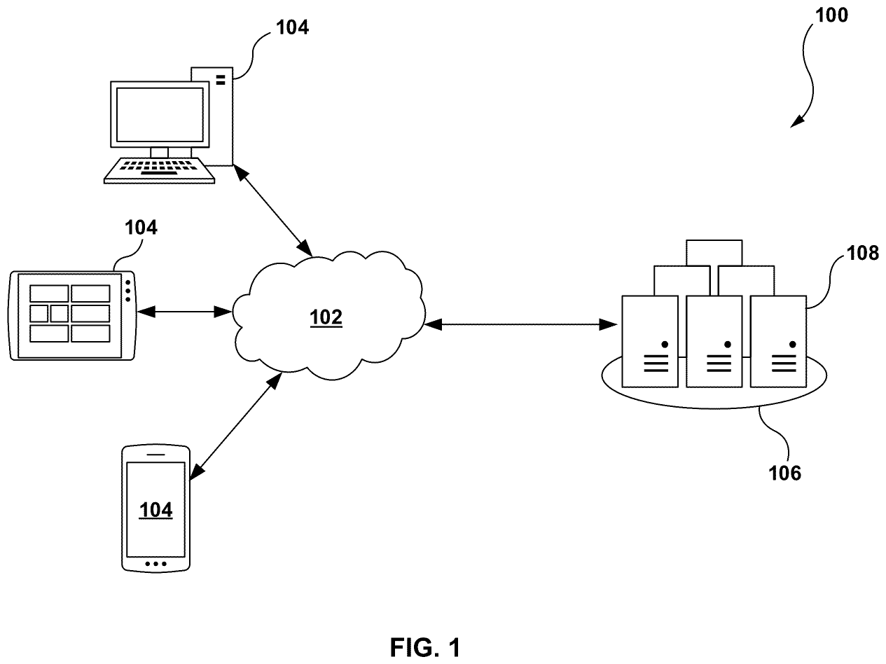

Referring now to FIG. 1, there is shown a computer network 100 that comprises an example embodiment of a system for processing medical diagnostic information. More particularly, the computer network 100 comprises a wide area network 102 such as the Internet to which various client devices 104 and data center 106 are communicatively coupled. The client devices 104 may be used by a clinician in furtherance of their medical practice. The data center 106 comprises a number of servers 108 networked together to collectively perform various computing functions. For example, in the context of a medical practice, the data center 106 may host online services in support of a medical practice and permit users (e.g. physicians or other clinicians) to log in to those servers using user accounts that give them access to various computer-implemented support services, such as electronic medical records and submission of billings.

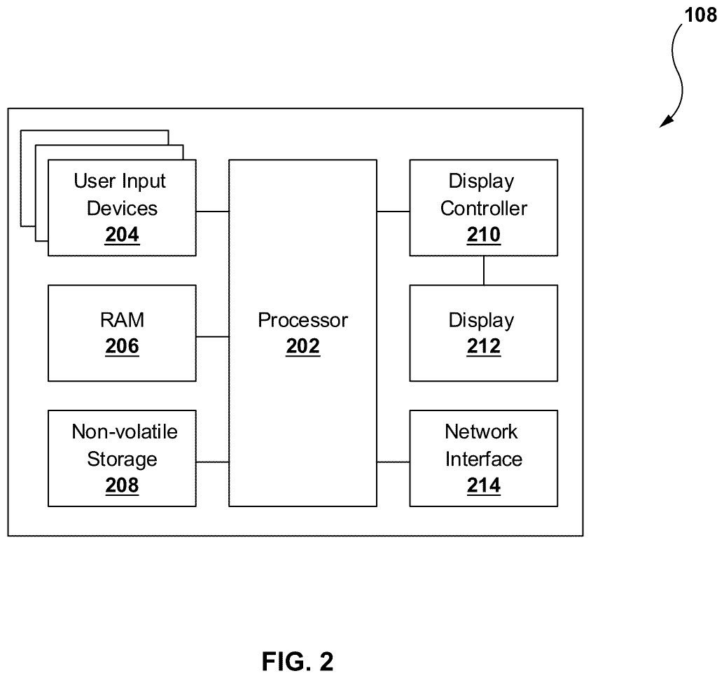

Referring now to FIG. 2, there is depicted an example embodiment of one of the servers 108 that comprises the data center 106. The server comprises a processor 202 that controls the overall operation of the server 108. The processor 202 is communicatively coupled to and controls several subsystems. These subsystems comprise user input devices 204, which may comprise, for example, any one or more of a keyboard, mouse, touch screen, voice control; random access memory (“RAM”) 206, which stores computer program code for execution at runtime by the processor 202; non-volatile storage 208, which stores the computer program code executed by the RAM 206 at runtime; a display controller 210, which is communicatively coupled to and controls a display 212; and a network interface 214, which facilitates network communications with the wide area network 102 and the other servers 108 in the data center 106. The non-volatile storage 208 has stored on it computer program code that is loaded into the RAM 206 at runtime and that is executable by the processor 202. When the computer program code is executed by the processor 202, the processor 202 causes the server 108 to implement a method for processing medical diagnostic information such as is described in more detail below. Additionally or alternatively, the servers 108 may collectively perform that method using distributed computing. While the system depicted in FIG. 2 is described specifically in respect of one of the servers 108, analogous versions of the system may also be used for the client devices 104.

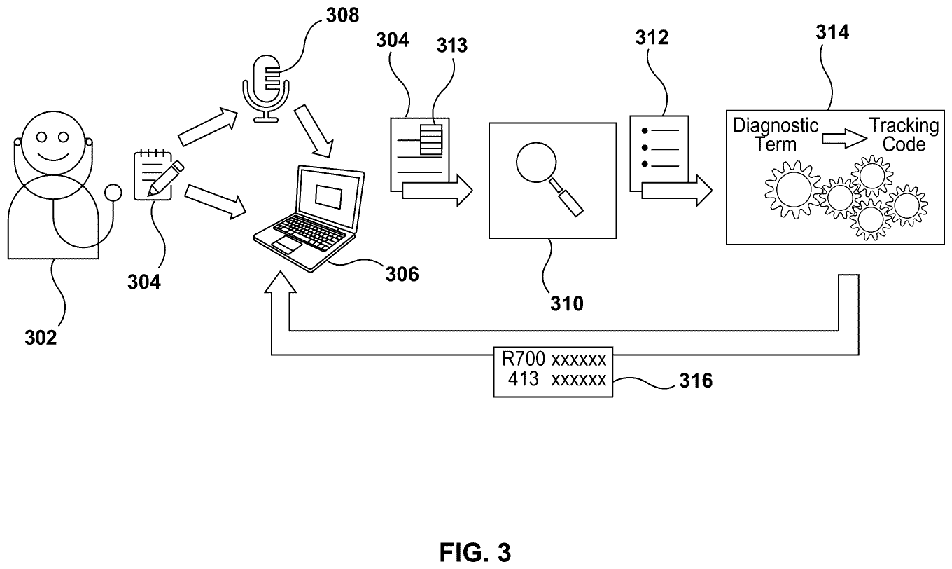

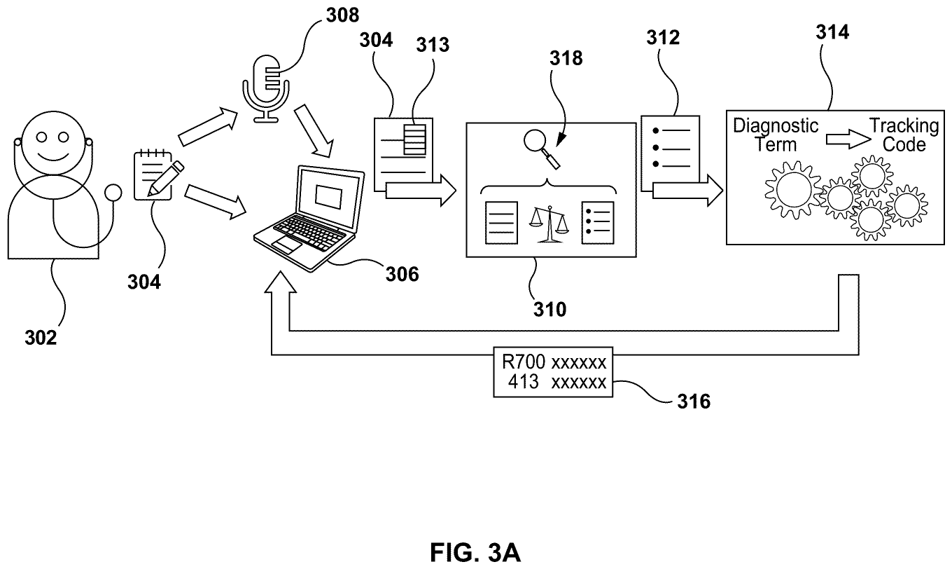

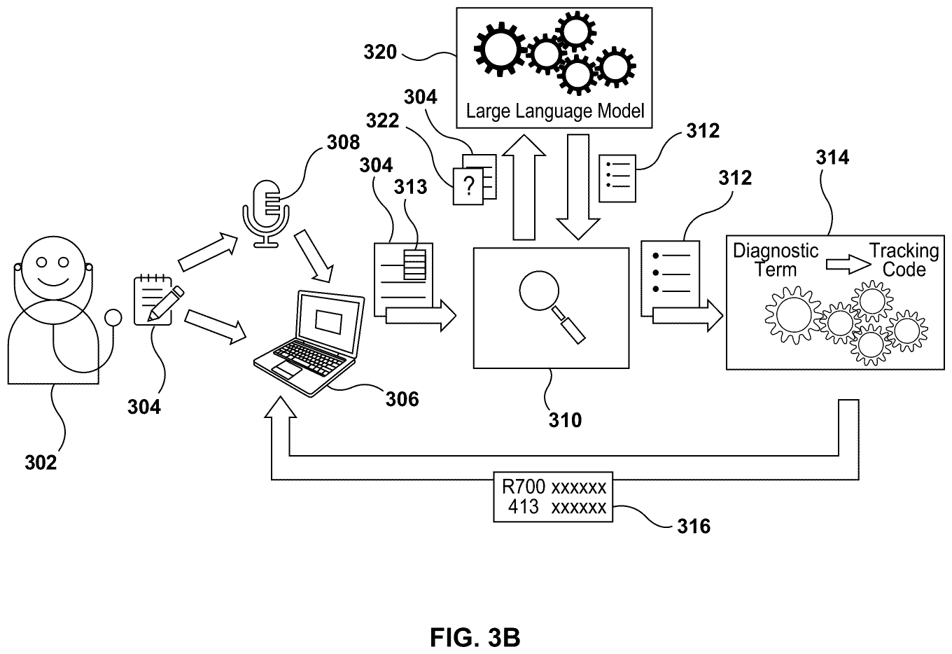

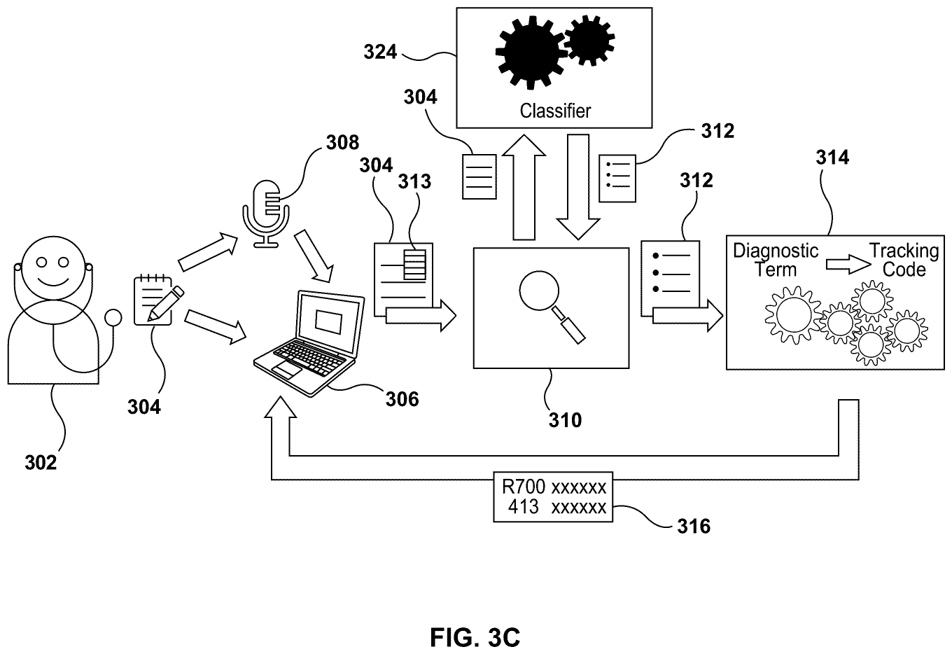

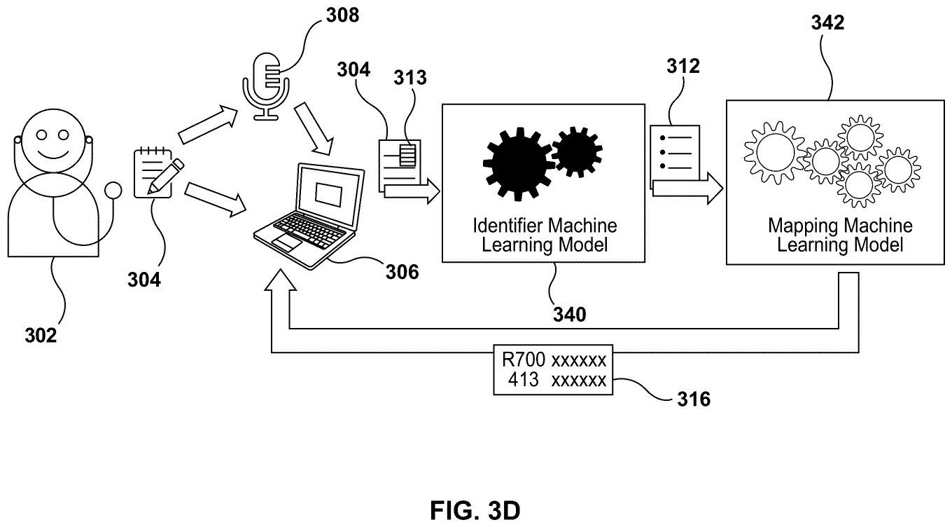

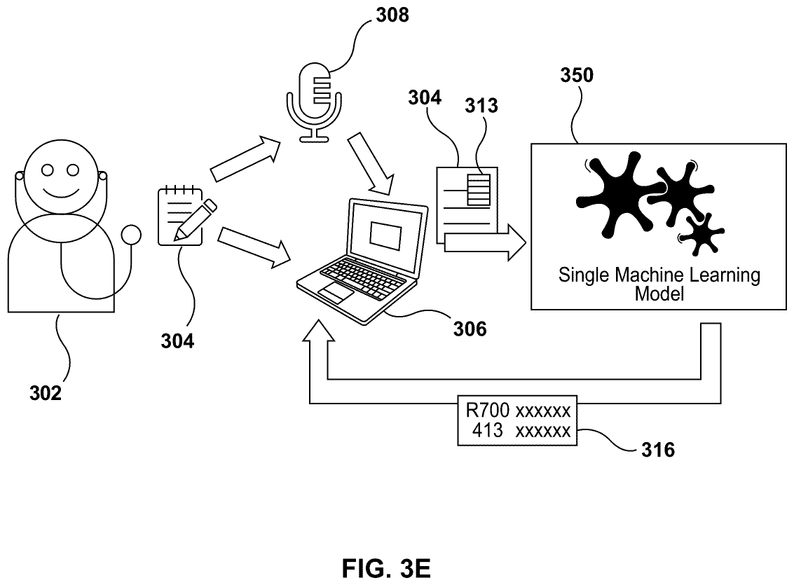

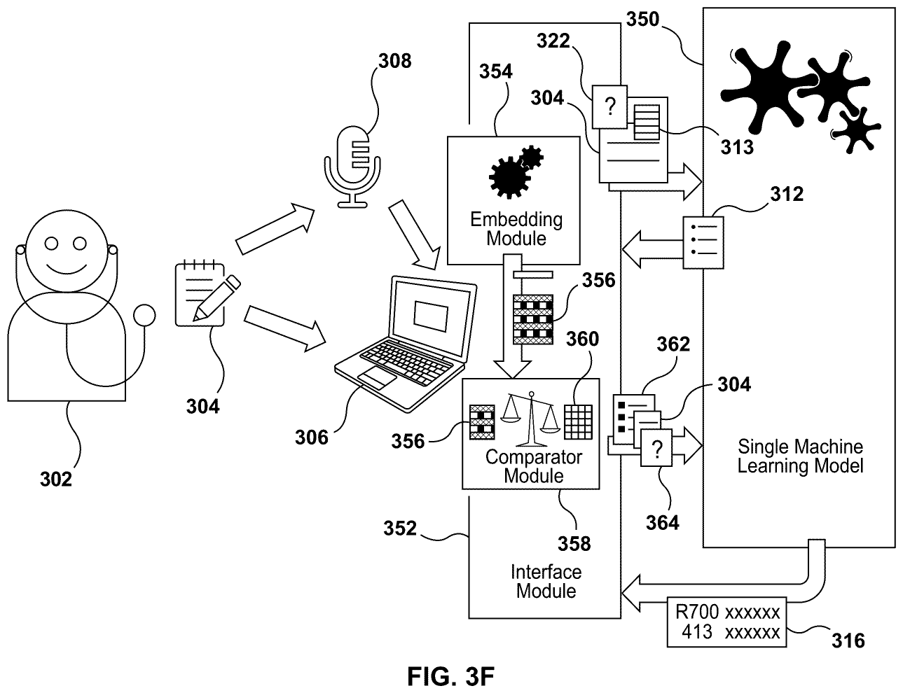

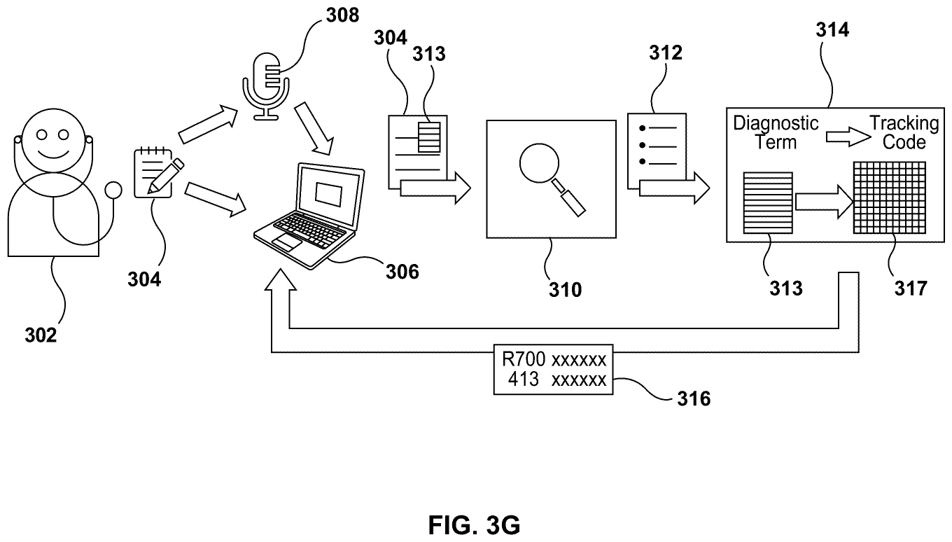

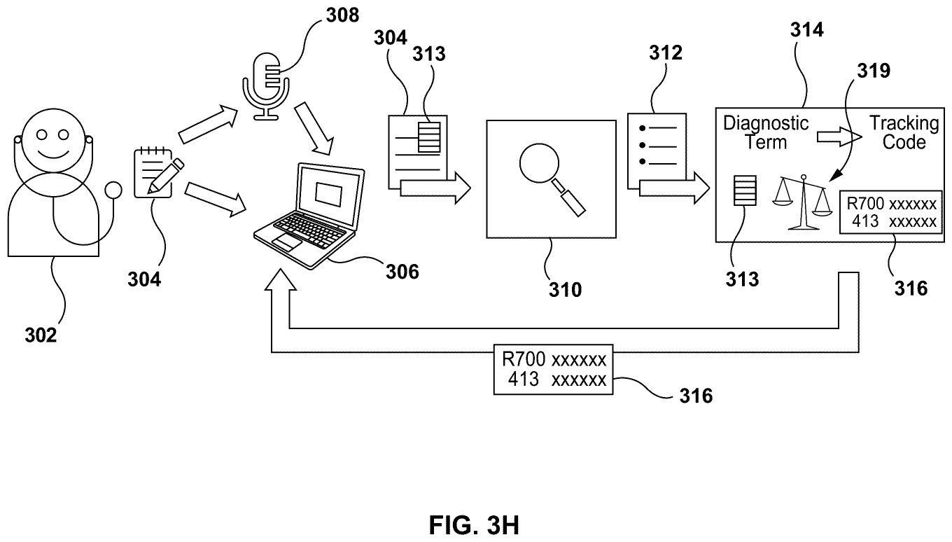

Reference is now made to FIG. 3, which shows an overview schematic representation of an illustrative embodiment of a computer implemented method for processing medical diagnostic information. A clinician 302 generates a text block 304 containing medical diagnostic information for a patient, for example, patient notes. Optionally, the text block 304 may also include the date and time of the relevant service(s). The clinician 302 may be, for example, a human physician, physician's assistant, nurse practitioner, or a nurse, or may be a veterinarian, or a veterinary technician. The foregoing are merely non-limiting examples.

The medical diagnostic information in the text block 304 will comprise diagnostic terms 313 (and will also typically comprise additional information). The phrase “diagnostic terms”, as used herein, refers to terms related to a diagnosis or medical consultation. “Diagnostic terms” include not only terms specifically describing a diagnosis, but also other steps associated with the consultation, including the steps taken to arrive at a diagnosis, differential diagnoses considered, prognosis, and treatment provided. Of note, terms may be “diagnostic terms” even if no specific diagnosis is reached during a consultation. Thus, “diagnostic terms” may include terms related to a physical examination which resulted in referral to a specialist, or referral for additional diagnostic tests, where the consulting practitioner was unable to arrive at a specific diagnosis. For example, initial symptoms of fever/chills, headache, muscle aches, nausea and diarrhea are consistent with several possible conditions, and a practitioner may refer the patient for blood tests without making a diagnosis.

Examples of steps taken to arrive at the diagnosis include, without limitation, radiography, cytology, urinalysis, ultrasound, MRI, CT scan, endoscopy, histology, and physical examination (including gross identification, auscultation and palpation) and words or phrases related to any of the foregoing would be considered “diagnostic terms”.

Differential diagnoses are the possible causes/conditions considered in arriving at one or more probable diagnoses. There may be a single definitive diagnosis, or the clinician 302 may not be able to arrive at a single definitive diagnosis for a variety of reasons (for example, the patient or animal owner may decline some tests, or test results may not yet be available). Terms associated with differential diagnoses would also be considered “diagnostic terms”.

A prognosis describes a projected outcome for the patient, and may be characterized, for example, as “good”, “poor”, or “guarded” (for example if some information needed for the prognosis is absent); these may also constitute “diagnostic terms”.

Examples of treatment include prescription/medication, advice, physiotherapy, chemotherapy, amputation, euthanasia (in the case of non-human veterinary patients), “medical assistance in dying” or MAiD (in the case of human patients, and of course only where permitted by law and only in compliance with such law), castration, hysterectomy, ovariohysterectomy/spay, neuter, splint, cast, and monitoring, among others. Terms associated with treatment are also “diagnostic terms”.

Returning to FIG. 3, the text block 304 may be generated, for example, by typing into a client device 306 to generate the text block directly. The client device 306 may be, for example, a desktop computer, a laptop computer, a tablet computer, or a smartphone. Any of the client devices 104 shown in FIG. 1 may be a client device 306, for example. Alternatively, the text block 304 may be a transcript of an audio stream containing spoken words received by a microphone 308 associated with the client device 306. The microphone 308 may be, for example, an inbuilt microphone of the client device 306, or an external microphone, including a microphone of a distinct device, such as the microphone of a smartwatch coupled to the client device 306. The audio stream may be transcribed locally on the client device 306, or may be sent for remote processing and then the resultant text block may be returned to the client device 306. In one embodiment, the transcription of text to speech may be performed using the Whisper open source software, which is available at the URL https://github.com/openai/whisper and is incorporated herein by reference. In another embodiment, the SpeechRecognition component of the Web Speech API, available at https://developer.mozilla.org/en-US/docs/Web/API/SpeechRecognition, may be used. The foregoing are merely illustrative examples and are not limiting. In another embodiment, the text block 304 may be obtained by optical character recognition (OCR) or similar technology, for example by writing on a screen of the client device 306 with a stylus, or by analyzing a scanned document such as handwritten notes. Preferably, the transcribed or OCR-generated text block 304 is presented to the clinician 302 to give the clinician 302 an opportunity to make corrections or changes.

The text block 304 is sent to an identification module 310, which obtains diagnostic term information 312 identifying the diagnostic terms 313 within the text block 304. The diagnostic term information 312 may consist of the diagnostic terms 313 themselves, for example a list of the diagnostic terms 313, or may comprise information identifying the diagnostic terms 313 (with or without the diagnostic terms 313 themselves). For example, the diagnostic term information 312 may comprise the text block 304 reproduced in a markup language with the diagnostic terms 313 flagged, or may comprise a list of codes that correspond to the diagnostic terms 313. The diagnostic term information 312 is then sent to a mapping module 314, which maps the identified diagnostic terms 313 to specific matching tracking codes 316 that can be used for billing, and then returns those specific matching tracking codes 316 for review by the clinician 302, who can use them to submit the appropriate claim for their fees.

In one embodiment, the identification module 310 may perform an initial semantic search for a given text block 304, followed by more sophisticated filtering at the mapping module 314.

The identification module 310 and/or the mapping module 314 may execute within the client device 306, or on one or more separate devices remote from the client device 306 (e.g. on one or more servers 108 in the data center 106 shown in FIG. 1). In the latter case, the text block 304, diagnostic term information 312 and/or matching tracking codes 316 may be transmitted via a wide area network such as the wide area network 102 shown in FIG. 1. Although not shown explicitly in FIG. 3, where the identification module 310 executes on device(s) remote from the client device 306, the diagnostic term information 312 may be returned from the identification module 310 to the client device 306. For example, where the identification module 310 and/or the mapping module 314 execute on remote device(s), the client device 306 may send the text block 304 to the identification module 310, receive the diagnostic term information 312 from the identification module 310, and then send the diagnostic term information 312 to the mapping module 314.

The identification module 310 and the mapping module 314 may be implemented in a variety of ways, some non-limiting illustrative examples of which are described further below.

In one embodiment, as shown in FIG. 3A, the identification module 310 may obtain the diagnostic term information 312 identifying the diagnostic terms 313 within the text block 304 by a comparison 318 of text strings (words or consecutive sets of words) within the text block 304 to a predetermined list of diagnostic terms. The comparison may be a pure text comparison. In such an arrangement, the predetermined list may be a list of complete diagnostic terms, or a list of etymological roots for the diagnostic terms, or a combination thereof. For example, the list may include both the terms “humerus” and “humeral” (complete diagnostic term), or may include only the root “humer”. As an alternative to a pure text comparison, the identification module 310 may compare text strings by generating text embeddings for the text strings in the text block 304 and compare those text embeddings to text embeddings for the diagnostic terms in the list. For example, and without limitation, the model “text-embedding-ada-002”, also referred to as “Ada-2”, offered by OpenAI and described at the website https://platform.openai.com/docs/guides/embeddings/what-are-embeddings and incorporated by reference herein, may be used.

In another embodiment, as shown in FIG. 3B, the identification module 310 may obtain the diagnostic term information 312 identifying the diagnostic terms 313 within the text block 304 by submitting the text block 304 to a trained large language model (LLM) 320 along with an engineered prompt 322 for the LLM 320 to return the diagnostic term information 312, and then receiving the returned diagnostic term information 312. In such an embodiment, the LLM 320 may be a general purpose LLM, or a bespoke or purpose-trained LLM. One non-limiting example of a general purpose LLM is ChatGPT-3.5 from OpenAI (https://openai.com/), having an address at 3180 18th Street, San Francisco, California 94110. The LLM 320 may be executing on a computer system that is remote from the computer system executing the identification module 310, or on the same computer system, which may be the client device 306. In the case of a bespoke or purpose-trained LLM, the LLM may form part of the identification module 310.

In a still further embodiment, as shown in FIG. 3C, the identification module 310 may obtain the diagnostic term information 312 identifying the diagnostic terms 313 within the text block 304 by submitting the text block 304 to a trained classifier 324, and receiving the diagnostic term information 312 from the classifier 324. As in the case of an LLM 320 (FIG. 3B), the classifier 324 may be executing on a computer system that is remote from the computer system executing the identification module 310, or on the same computer system (which may be the client device 306), and may form part of the identification module 310.

As noted above, in each of the embodiments shown in FIGS. 3 through 3C, the identification module 310 passes the diagnostic term information 312 to a mapping module 314 that maps the diagnostic terms 313 identified by the diagnostic term information 312 to corresponding respective tracking codes to identify matching tracking codes 316 that correspond to the identified diagnostic terms 313. The mapping module 314 then returns the matching tracking codes 316, for example as a list displayed on the client device 306. The tracking codes will at least include service codes. Service codes are the codes used to facilitate payment to the clinician, for example by a government agency or an insurance company. Typically, a service code will comprise a numeric, alphabetic or alphanumeric code and will have an associated description or title, and may also have an associated set fee (or fees for each professional if there is more than one professional involved in providing the service). Some illustrative, non-limiting examples of service codes from the Ontario Health Insurance Plan (OHIP) are listed below (with fees omitted):

| Service | |
|---|---|
| Code | Description |
| G382 | Supervision of chemotherapy (pharmacologic therapy of |
| | malignancy or autoimmune disease) by telephone, monthly |
| Z721 | Pharmacological suppression of premature labour by I.V. |
| | therapy to be claimed once per physician after 3 hours of |
| | supervision in same institution |
| R700 | With hypothermia and without bypass - basic fee for |
| | cardiovascular procedures |

In some contexts, a service code may be sufficient for facilitating payment. In other contexts, both a service code and a diagnostic code may be required. In such contexts, the tracking codes may comprise both service codes and diagnostic codes. Some illustrative, non-limiting examples of diagnostic codes from OHIP are listed below:

| Diagnostic | |
|---|---|
| Code | Description |
| 162 | Malignant Neoplasms: Bronchus, lung |
| 640 | Threatened abortion, haemorrhage in early pregnancy |
| 413 | Ischaemic and other forms of heart disease: acute coronary |
| | insufficiency, angina pectoris, acute ischaemic heart disease |

In some cases, there may also be additional codes, for example there may be a modifier code or adjustment code based on the manner in which the service is provided (e.g. the fee may be reduced by a set percentage for a telephone or online consultation) or, in a veterinary context, a modifier for the type of animal; these are also considered to be tracking codes.

The mapping module 314 may use a number of techniques to map the diagnostic terms 313 identified by the diagnostic term information 312 to the corresponding respective tracking codes.

In a first embodiment, as shown in FIG. 3G, the mapping module 314 compares the diagnostic terms 313 identified by the diagnostic term information 312 to a correspondence table 317 (or equivalently a lookup table) in which ones of the diagnostic terms 313 correspond to respective ones of the tracking codes.

In another embodiment, the mapping module 314 calculates diagnostic term text embeddings for the diagnostic terms identified by the diagnostic term information 312. The mapping module 314 then compares the diagnostic term text embeddings to tracking code text embeddings for the tracking codes to identify those of the tracking codes whose respective tracking code text embeddings most closely match the diagnostic term text embeddings. One implementation of such an embodiment is shown in FIG. 3F, described further below. As can be seen above, the tracking codes (service codes and diagnostic codes) may have associated descriptive text, and descriptive text embeddings may also be created and included in the tracking code text embeddings to facilitate the comparison. Since text embeddings are vectors, comparing the diagnostic term text embeddings to the tracking code text embeddings may be achieved, for example, by comparison of mathematical distance between vectors. For example, and without limitation, cosine similarity may be used to compute the mathematical difference between the vectors.

Preferably, in such an embodiment the tracking code text embeddings (including any descriptive text embeddings) are pre-calculated for improved performance, and when the system is initialized it will first check that all tracking code text embeddings are up to date and accurate. In one embodiment, a database containing predetermined text embeddings for tracking codes, along with the actual tracking code title, is provided. Whenever a tracking code title is updated (for example by a new release of the code documentation) the corresponding text embeddings can be updated for the new title. Optionally, the tracking code titles in the database can be compared to the current tracking code title (e.g. daily), and if the current published tracking code title has changed, the text embeddings for that tracking code title can be recalculated and saved.

In some embodiments, both with or without the use of text embeddings, mapping the identified diagnostic terms to the corresponding respective tracking codes comprises mapping by semantic similarity 319, as shown in FIG. 3H. Semantic similarity measures the similarity of content or meaning, as opposed to measuring similarity in structural or lexicographical terms. For example, the semantic similarity of the diagnostic terms 313 identified by the diagnostic term information 312 to the descriptions associated with respective tracking codes 316 may be assessed. By way of non-limiting example, the word “ventricular” would be semantically closer to the description “With hypothermia and without bypass basic fee for cardiovascular procedures” for the service code R700 than to the descriptions for either the service code G382 (“Pharmacological suppression of premature labour . . . ”) or Z721 (“Supervision of chemotherapy . . . ”). This is because despite the fact that the words themselves are structurally and lexically different, “ventricular” is semantically similar to “cardiovascular” (both relate to the heart). Techniques for comparison of semantic similarity are well-known in the art, see for example https://www.geeksforgeeks.org/different-techniques-for-sentence-semantic-similarity-in-nlp/incorporated herein by reference, and can be implemented by one of ordinary skill in the art, now informed by the present disclosure.

Optionally, the use of text embeddings and/or semantic similarity may be combined with the use of an LLM. A first set of tracking codes may be obtained using text embeddings and/or semantic similarity and then passed to a trained LLM along with some or all of the text block, and the LLM can then return a refined set of tracking codes. In some embodiments, the text block may be passed to an LLM only if using text embeddings and/or semantic similarity fails to identify tracking codes with sufficient confidence scores. If the confidence scores for the semantic search are too low, an LLM may be prompted to look at the tracking codes which were most closely related to the text block, and return the best matching code.

In other embodiments, the mapping module 314 may use a trained machine learning engine to map the diagnostic terms 313 identified by the diagnostic term information 312 to the corresponding tracking codes to obtain matching tracking codes 316. For example, the mapping module 314 may provide the diagnostic term information 312 to a trained classifier, and receive the matching tracking codes 316 from the trained classifier, or may provide the diagnostic term information 312 to a trained LLM along with a suitable engineered prompt requesting the matching tracking codes 316, and receive the matching tracking codes from the trained LLM.

FIG. 3D shows an embodiment in which, after receiving a text block 304 containing diagnostic terms 313, an identifier machine learning model 340 is used to obtain the diagnostic term information 312 identifying the diagnostic terms 313 within the text block 304. The diagnostic term information 312 is then passed to a mapping machine learning model 342 to map the diagnostic terms 313 to corresponding respective tracking codes to obtain the matching tracking codes 316, which are then returned to the client device 306. As can be seen in FIG. 3D, in the illustrated embodiment the identifier machine learning model 340 and the mapping machine learning model 342 are separate, distinct, individual models. The identifier machine learning model 340 may be a classifier, and the mapping machine learning model 342 may be a large language model, for example. The identifier machine learning model 340 may be executing on a computer system that is remote from the computer system executing the mapping machine learning model 342, or on the same computer system, which may be the client device 306.

FIG. 3E shows an embodiment in which, after receiving a text block 304 containing diagnostic terms 313, a single trained machine learning model 350 is used to identify the matching tracking codes 316 that correspond to the diagnostic terms 313 within the text block 304. The single trained machine learning model 350 may be executing on a computer system that is remote from the client device 306, or on the client device 306.

FIG. 3F shows a particular implementation of the embodiment in FIG. 3E in which the single trained machine learning model 350 subsumes functions of the identification module and the mapping module, and in which the single trained machine learning model 350 is an LLM. For example and without limitation, the single trained machine learning model may be the ChatGPT-3.5 LLM. In the embodiment shown in FIG. 3F, an interface module 352 executes on the client device 306, and handles communication with the single trained machine learning model 350. Analogously to the approach described in FIG. 3B, the interface module 352 submits the text block 304 to the single trained machine learning model 350 along with an engineered prompt 322 for the single trained machine learning model 350 to return the diagnostic term information 312, and then receives the returned diagnostic term information 312. The interface module 352 communicates the diagnostic terms 313 identified by the diagnostic term information 312 to an embedding module 354 also executing on the client device 306, which calculates diagnostic term text embeddings 356 for those diagnostic terms. The diagnostic term text embeddings 356 are communicated to a comparator module 358 also executing on the client device 306. The comparator module 358 compares the diagnostic term text embeddings 356 to tracking code text embeddings 360 for the tracking codes, for example using mathematical distance such as cosine similarity, to identify as candidate tracking codes 362 those of the tracking codes whose respective tracking code text embeddings 360 most closely match the diagnostic term text embeddings 356. A score can be created and the comparator module 358 can sort the identified tracking codes according to the score to obtain the candidate tracking codes 362. The interface module 352 sends the highest ranked candidate tracking codes 362 (e.g. the top 5) back to the single trained machine learning model 350 along with the text block 304 and another engineered prompt 364 to narrow down the candidate tracking codes 362. The single trained machine learning model 350 then returns the matching tracking codes 316 to the interface module 352. Note that in other embodiments, the embedding module 354 and/or the comparator module 358 may execute on device(s) remote from the client device 306.

Notably, the embodiment shown in FIG. 3F is not merely sending a text block to a machine learning model and asking that machine learning model to identify tracking codes that correspond to diagnostic terms within the text block. The embodiment shown in FIG. 3F leverages the single trained machine learning model 350 at two different stages. These stages are separated by a step executed outside of the machine learning model 350, namely comparing the diagnostic term text embeddings 356 to tracking code text embeddings 360 to identify those of the tracking codes whose respective tracking code text embeddings 360 most closely match the diagnostic term text embeddings 356. Thus, the embodiment shown in FIG. 3F performs specific processing on a first result (diagnostic term information 312) from the machine learning model 350, which processing is used to generate information (candidate tracking codes 362) used to obtain a second result (matching tracking codes 316).

In embodiments in which a machine learning model is used to map identified diagnostic terms to corresponding respective tracking codes, or to otherwise identify the tracking codes that correspond to the diagnostic terms within the text block, the machine learning model may be trained with the relevant corpus of tracking codes, or may be provided with (or with access to) the relevant schedule of tracking codes.

In some cases, the clinician 302 may know (or think they know) one or more of the tracking codes, and may include these tracking code(s) within the text block 304 (e.g. the clinician may say “bill for code G382”). Thus, preferably the system is configured to identify specific instances of the tracking codes within the text block 304 and ensure that those tracking codes within the text block 304 are also returned to the clinician 302. This may be achieved in a number of ways. For example, direct text matching may be used, such as by scanning the text block 304 for tracking codes before sending the text block 304 to the identification module 310, or by having the identification module 310 include explicit tracking codes within the ambit of diagnostic terms. The identification module 310 can then return any such tracking codes directly, or pass them to the mapping module 314 along with (or as part of) the diagnostic term information 312, and the mapping module 314 can treat explicit tracking codes as a special case that maps to itself. For example, if the clinician 302 includes the service code “Z721” in the text block 304, the service code “Z721” can be identified within the diagnostic term information 312 to be passed to the mapping module 314, which maps the service code “Z721” to itself and returns service code “Z721” along with any other matching tracking codes 316 mapped to the diagnostic terms 312. Optionally, where the mapping module 314 receives a tracking code as part of the diagnostic term information 312, the mapping module 314 may map that tracking code to similar tracking codes in case of an error by the clinician.

The matching tracking codes 316 that are returned by the mapping module 314 will be those that best correspond to the diagnostic terms 313, and may be, for example, the five (5) or ten (10) best matches (e.g. the five (5) or ten (10) best service codes and/or the five (5) or ten (10) best diagnostic codes). The clinician 302 can then select the appropriate tracking codes from those returned (e.g. from a pull-down menu) and these can be used to populate an online billing form, along with the date and time if provided in the text block 304. Thus, a system according to the present disclosure may be integrated with an online claim/billing submission system. A clinician 302 can also manually enter tracking codes that were not returned by the system. Optionally, tracking codes that do not meet a confidence threshold may not be returned, even if otherwise highly ranked. Also optionally, a machine learning model using reinforcement learning may be used to improve the accuracy of the returned matching tracking codes 316, using data from the text blocks 304 and the selections and/or corrections made by the clinicians 302.

Because the system described herein can process the medical diagnostic information in patient notes to generate the tracking codes needed for billing, the system can substantially automate the billing process for the clinician 302 so that it occurs substantially simultaneously upon entry of the patient notes as a text block 304. Consequently, the billing process can be integrated into the existing clinical process of creating patient notes, with tracking codes being recommended when the care provided is fresh in the mind of the clinician 302, such that very little additional time or effort is required for the billing process.

In some embodiments, the system may include a frontend to provide the interface used by the clinician 302, and a backend which provides the functionality implemented by the identification module 310 and the mapping module 314. The frontend may be implemented as a React-based web application implemented using the React JavaScript Library (https://react.dev/) and executing on the client device 102, 306, for example using the Next.js framework (https://nextjs.org/). The backend may include a Flask application programming interface (API) implementation (https://flask.palletsprojects.com/en/3.0.x/) that communicates with the frontend as well as with other aspects of the system, such as one or more third-party machine learning systems and one or more databases. The database may be implemented, for example, as a Structured Query Language (SQL) database, for example using PostgreSQL (https://www.postgresql.org/). The foregoing architecture description is merely illustrative, and is not intended to be limiting.

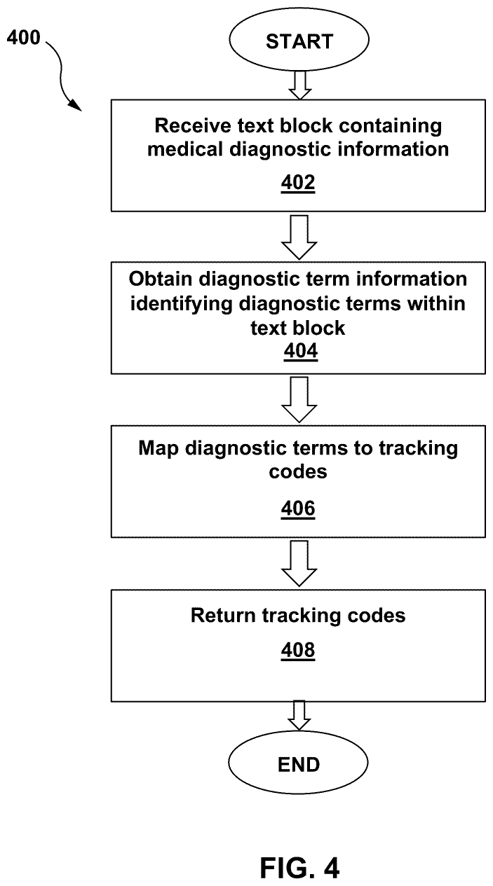

Reference is now made to FIG. 4, which is a flow chart showing an illustrative method 400 for processing medical diagnostic information. At step 402, the method 400 receives a text block containing medical diagnostic information, and at step 404, the method 400 obtains diagnostic term information identifying diagnostic terms within the text block. Step 404 may be carried out using any of the techniques described above. At step 406, the method 400 maps the diagnostic terms identified by the diagnostic term information obtained at step 404 to corresponding respective tracking codes to identify matching tracking codes that correspond to the identified diagnostic terms. Step 406 may be carried out, for example, using any of the techniques described above. At step 408, the method 400 returns the matching tracking codes identified at step 406.

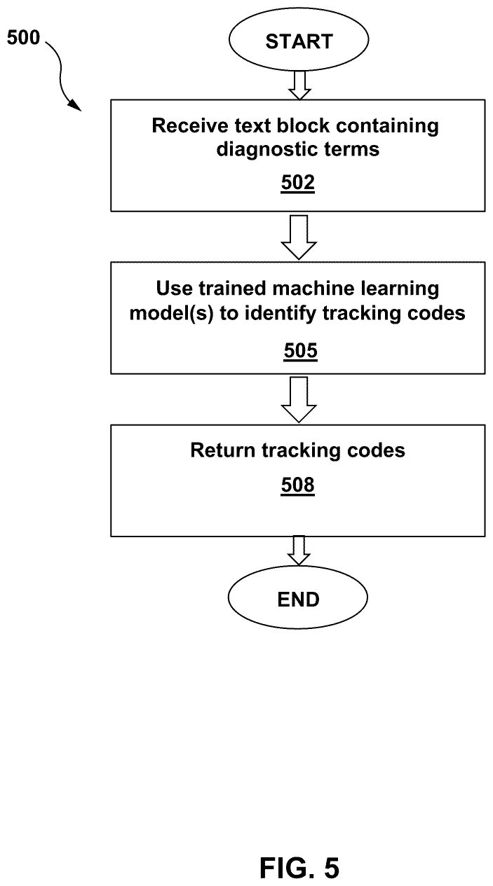

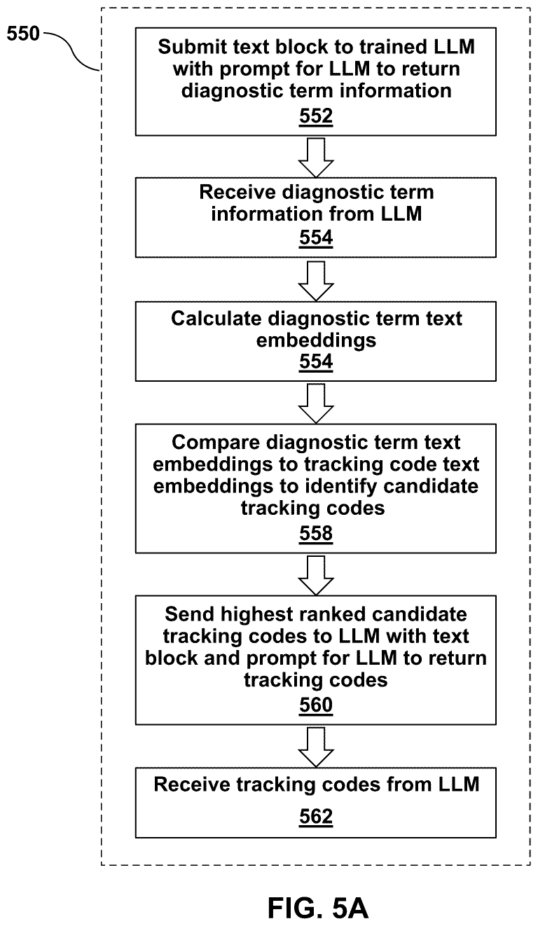

FIG. 5 is a flow chart showing another illustrative method 500 for processing medical diagnostic information. At step 502, the method 500 receives a text block containing diagnostic terms, and at step 505, the method 500 uses at least one trained machine learning model to identify matching tracking codes that correspond to the diagnostic terms. At step 508, the method 500 returns the matching tracking codes.

In some embodiments of the method 500, step 505 may use an identifier machine learning model 340, such as an LLM or a classifier, to identify diagnostic terms within the text block. In some embodiments of the method 500, step 505 may use a mapping machine learning model 342, such as an LLM or a classifier, to map the diagnostic terms to corresponding respective tracking codes. In a particular embodiment that is equivalent to the method 400 shown in FIG. 4, step 505 may use an identifier machine learning model 340 to obtain diagnostic term information identifying the diagnostic terms within the text block (step 404) and use a mapping machine learning model 342 to map the diagnostic terms to the corresponding respective tracking codes (step 406). In an alternative embodiment of the method 500, step 505 uses a single trained machine learning model 350 to identify the matching tracking codes that correspond to the diagnostic terms, as shown in FIG. 3E.

FIG. 5A is a flow chart showing a particular implementation of step 505 of the method 500 which corresponds to the schematic illustration shown in FIG. 3F; that is, a particular implementation of the use of a single trained machine learning model 350 to identify the matching tracking codes that correspond to the diagnostic terms. Thus, FIG. 5A, is an expanded illustrative implementation of step 505 to show an illustrative method 550 of using a trained machine learning model, and in particular a LLM, to identify matching tracking codes that correspond to the diagnostic terms.

At step 552, the method 550 submits the text block to a trained LLM with a first prompt for the LLM to return the diagnostic term information. At step 554, the method 550 receives the diagnostic term information from the LLM. At step 556, the method 550 calculates diagnostic term text embeddings for the diagnostic terms identified by the diagnostic term information received from the LLM, and at step 558 the method 550 compares the diagnostic term text embeddings to tracking code text embeddings for the tracking codes to identify as candidate tracking codes those of the tracking codes whose respective tracking code text embeddings most closely match the diagnostic term text embeddings. At step 560, the method 550 sends the highest ranked candidate tracking codes (e.g. the top five (5), ten (10), fifteen (15) or twenty (20) candidate tracking codes) to the LLM along with the text block and an engineered prompt for the LLM to return the matching tracking codes, and at step 562 the method 550 receives the matching tracking codes from the LLM.

The text block received at steps 402 or 502 may be a transcript of an audio stream containing spoken words received by a microphone of a data processing system, or may be a typed text block, or a text block obtained by OCR.

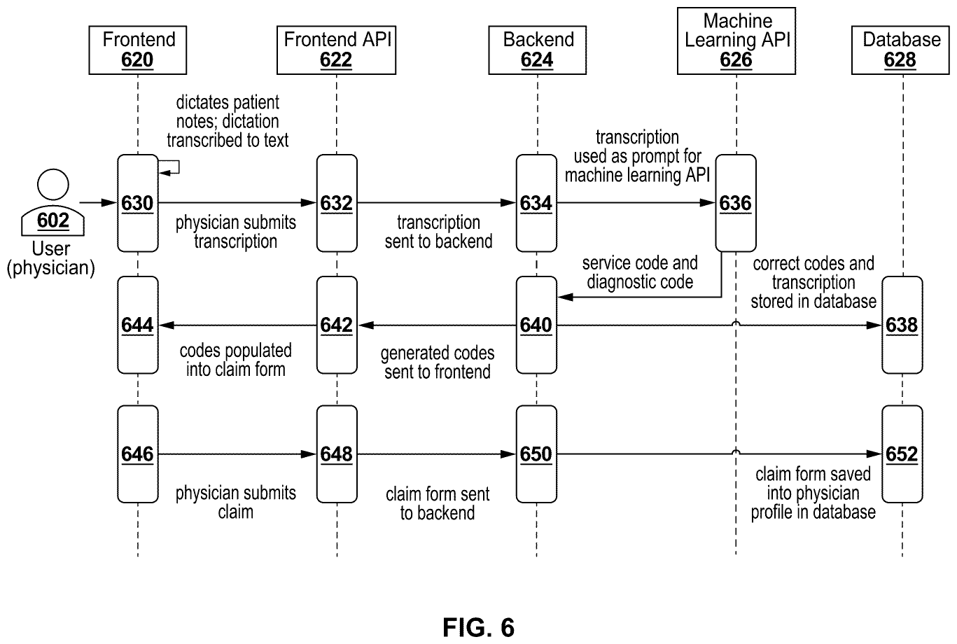

Reference is now made to FIG. 6, which is a process flow diagram for an illustrative implementation of the method 500 in which step 505 uses a single trained machine learning model 350 in the form of an LLM to identify the matching tracking codes that correspond to the diagnostic terms. Along with a user (clinician) 602, aspects of the system are shown in vertical columns, including a frontend 620, a frontend API 622, a backend 624, a machine learning model API 626, and a database 628. The frontend API 622 may be a Flask API, as noted above, and facilitates communication between the frontend 620 and the backend 624. The database 628 may be, but is not limited to, a PostgreSQL database. The machine learning model API 626 facilitates communication between the backend 624 and the machine learning model that implements step 505, which may be, but is not limited to, a third party machine learning model, such as ChatGPT-3.5 offered by OpenAI.

The user 602 submits a text block (e.g. text block 304 in FIG. 3E), optionally obtained by transcription of audio, to the frontend 620. At step 630, the frontend 620 transmits the text block, via the frontend API 622 at step 632, to the backend 624. At step 634, the backend 624 submits the text block, along with a suitable engineered prompt, to the machine learning model API 626. After processing by the machine learning model, the machine learning model API 626 returns the matching tracking codes to the backend 624 at step 636. The backend 624 then transmits the text block and the matching tracking codes to the database 628 for storage at step 638, and returns the matching tracking codes to the frontend API 622 at step 640. The frontend API 622 then transmits the matching tracking codes at step 642 to populate a web-based claim form in the frontend 620 at step 644. After review and possible editing by the user 602, at the direction of the user 602 the frontend 620 transmits the claim form at step 646. The frontend API 622 passes the claim form to the backend 624 at step 648, and at step 650 the backend 624 sends the completed claim form to the database 628 for storage at step 652. Formal submission of the claim form stored on the database 628 for reimbursement may be implemented by a separate claim submission system, for example a claim submission system implemented by a government body or an insurance company.

Optionally, systems and methods according to the present disclosure may be integrated into electronic medical record (EMR) systems, including those adhering to the Fast Healthcare Interoperability Resources (FHIR) standard. For example, the text blocks (e.g. text block 304) may be entered into the EMR system as patient notes, or the system can harvest the text blocks from the patient notes in the EMR. In one embodiment, an API may be provided to enable EMR platforms send patient notes to servers implementing one of the above-described methods, and receive the associated matching tracking codes in response. Optionally, systems according to the present disclosure can integrate with a clinician's billing history, so that the reinforcement learning components can be pre-trained based on consideration of the most common service codes used by a specific clinician. The systems can also optionally take account of a patient's medical history, allowing the recommended service codes to be based at least in part on consideration of a patient's past conditions. For instance, if a patient has already been seen by an internal medicine physician, then in a subsequent visit the physician is likely going to simply bill for a follow up visit. The system could identify these situations and make appropriate billing code recommendations when a specific patient is selected.

In preferred embodiments, implementations of systems, methods and computer program products are configured for compliance with relevant privacy legislation, with suitable security and encryption protocols to protect confidential patient information, and implement privacy-by-design.

As can be seen from the above description, the technology described herein represents significantly more than merely using categories to organize, store and transmit information and organizing information through mathematical correlations. The technology is in fact an improvement to medical clinical technology, as it provides a technical solution to improve the efficiency with which physicians can identify the appropriate service codes and diagnostic codes. This facilitates the benefit of enabling physicians to spend more time providing critical medical care to the patients who need it most. Consequently, the technology described herein is confined to medical (including veterinary) applications.

The processor used in the foregoing embodiments may comprise, for example, a processing unit (such as a processor, microprocessor, or programmable logic controller) or a microcontroller (which comprises both a processing unit and a non-transitory computer readable medium). Examples of computer readable media that are non-transitory include disc-based media such as CD-ROMs and DVDs, magnetic media such as hard drives and other forms of magnetic disk storage, semiconductor based media such as flash media, random access memory (including DRAM and SRAM), and read only memory. As an alternative to an implementation that relies on processor-executed computer program code, a hardware-based implementation may be used. For example, an application-specific integrated circuit (ASIC), field programmable gate array (FPGA), system-on-a-chip (SoC), or other suitable type of hardware implementation may be used as an alternative to or to supplement an implementation that relies primarily on a processor executing computer program code stored on a computer medium.

The embodiments have been described above with reference to flow, sequence, and block diagrams of methods, apparatuses, systems, and computer program products. In this regard, the depicted flow, sequence, and block diagrams illustrate the architecture, functionality, and operation of implementations of various embodiments. For instance, each block of the flow and block diagrams and operation in the sequence diagrams may represent a module, segment, or portion of code, which comprises one or more executable instructions for implementing the specified action(s). In some alternative embodiments, the action(s) noted in that block or operation may occur out of the order noted in those figures. For example, two blocks or operations shown in succession may, in some embodiments, be executed substantially concurrently, or the blocks or operations may sometimes be executed in the reverse order, depending upon the functionality involved. Some specific examples of the foregoing have been noted above but those noted examples are not necessarily the only examples. Each block of the flow and block diagrams and operation of the sequence diagrams, and combinations of those blocks and operations, may be implemented by special purpose hardware-based systems that perform the specified functions or acts, or combinations of special purpose hardware and computer instructions.

The terminology used herein is for the purpose of describing particular embodiments only and is not intended to be limiting. Accordingly, as used herein, the singular forms “a”, “an”, and “the” are intended to include the plural forms as well, unless the context clearly indicates otherwise (e.g., a reference in the claims to “a training data set” or “the training data set” does not exclude embodiments in which multiple training data sets are used). It will be further understood that the terms “comprises” and “comprising”, when used in this specification, specify the presence of one or more stated features, integers, steps, operations, elements, and components, but do not preclude the presence or addition of one or more other features, integers, steps, operations, elements, components, and groups. Directional terms such as “top”, “bottom”, “upwards”, “downwards”, “vertically”, and “laterally” are used in the following description for the purpose of providing relative reference only, and are not intended to suggest any limitations on how any article is to be positioned during use, or to be mounted in an assembly or relative to an environment. Additionally, the term “connect” and variants of it such as “connected”, “connects”, and “connecting” as used in this description are intended to include indirect and direct connections unless otherwise indicated. For example, if a first device is connected to a second device, that coupling may be through a direct connection or through an indirect connection via other devices and connections. Similarly, if the first device is communicatively connected to the second device, communication may be through a direct connection or through an indirect connection via other devices and connections. The term “and/or” as used herein in conjunction with a list means any one or more items from that list. For example, “A, B, and/or C” means “any one or more of A, B, and C”.

It is contemplated that any part of any aspect or embodiment discussed in this specification can be implemented or combined with any part of any other aspect or embodiment discussed in this specification.

The scope of the claims should not be limited by the embodiments set forth in the above examples, but should be given the broadest interpretation consistent with the description as a whole.

It should be recognized that features and aspects of the various examples provided above can be combined into further examples that also fall within the scope of the present disclosure. In addition, the figures are not to scale and may have size and shape exaggerated for illustrative purposes.

What is claimed is:

1. A computer-implemented method for processing medical diagnostic information, comprising:
obtaining diagnostic term information identifying diagnostic terms within a text block containing medical diagnostic information;
mapping the identified diagnostic terms to corresponding respective tracking codes to identify matching tracking codes that correspond to the identified diagnostic terms; and
returning the matching tracking codes.

2. The method of claim 1, wherein the tracking codes comprise at least service codes.

3. The method of claim 2, wherein the tracking codes further comprise diagnostic codes.

4. The method of claim 1, further comprising:
identifying specific instances of the tracking codes within the text block;
wherein the returned matching tracking codes include the specific instances of the tracking codes.

5. The method of claim 1, wherein obtaining the diagnostic term information identifying the diagnostic terms within the text block comprises comparing text strings within the text block to a predetermined list of diagnostic terms.

6. The method of claim 5, wherein comparing the text strings within the text block to the predetermined list of diagnostic terms comprises comparing text embeddings for the text strings to text embeddings for the predetermined list of diagnostic terms.

7. The method of claim 1, wherein obtaining the diagnostic term information identifying the diagnostic terms within the text block comprises:
submitting the text block to a trained large language model (LLM) with a first prompt for the LLM to return the diagnostic term information; and
receiving the diagnostic term information from the LLM.

8. The method of claim 7, wherein mapping the identified diagnostic terms to the corresponding respective tracking codes to identify the matching tracking codes comprises:
calculating diagnostic term text embeddings for the identified diagnostic terms; and
comparing the diagnostic term text embeddings to tracking code text embeddings for the tracking codes to identify as candidate tracking codes those of the tracking codes whose respective tracking code text embeddings most closely match the diagnostic term text embeddings.

9. The method of claim 8, wherein mapping the identified diagnostic terms to the corresponding respective tracking codes to identify the matching tracking codes further comprises:
sending highest ranked ones of the candidate tracking codes to the LLM along with the text block and a second prompt for the LLM to return the matching tracking codes; and
receiving the matching tracking codes from the LLM.

10. The method of claim 1, wherein obtaining the diagnostic term information identifying the diagnostic terms within the text block comprises:
submitting the text block to a trained classifier; and
receiving the diagnostic term information from the classifier.

11. The method of claim 1, wherein the text block is a transcript of an audio stream containing spoken words.

12. The method of claim 1, wherein mapping the identified diagnostic terms to the corresponding respective tracking codes comprises:
calculating diagnostic term text embeddings for the identified diagnostic terms; and
comparing the diagnostic term text embeddings to tracking code text embeddings for the tracking codes to identify those of the tracking codes whose respective tracking code text embeddings most closely match the diagnostic term text embeddings;
wherein the returned matching tracking codes include those of the tracking codes whose respective tracking code text embeddings most closely match the diagnostic term text embeddings.

13. The method of claim 12, wherein the tracking code text embeddings are pre-calculated.

14. The method of claim 12, wherein mapping the identified diagnostic terms to the corresponding respective tracking codes comprises mapping by semantic similarity.

15. The method of claim 12, wherein comparing the diagnostic term text embeddings to the tracking code text embeddings for the tracking codes comprises comparison by mathematical distance between vectors.

16. The method of claim 1, wherein mapping the identified diagnostic terms to the corresponding respective tracking codes comprises:
comparing the identified diagnostic terms to a correspondence table wherein ones of the diagnostic terms correspond to respective ones of the tracking codes.

17. The method of claim 1, wherein mapping the identified diagnostic terms to the corresponding respective tracking codes comprises:
providing the diagnostic term information to a trained classifier, and
receiving the matching tracking codes from the trained classifier.

18. The method of claim 1, wherein mapping the identified diagnostic terms to the corresponding respective tracking codes comprises:
providing the diagnostic term information to a trained large language model (LLM) along with a prompt requesting the matching tracking codes; and
receiving the matching tracking codes from the trained LLM.

19. A data processing system comprising at least one processor and memory coupled to the at least one processor, wherein the memory contains instructions which, when executed by the at least one processor, cause the at least one processor to carry out a method according to claim 1.

20. A computer program product comprising at least one tangible non-transitory computer-readable medium containing instructions which, when executed by at least one processor of a data processing system, cause the data processing system to carry out a method according to claim 1.

21. A method for processing medical diagnostic information, comprising:
receiving a text block containing diagnostic terms;
using at least one trained machine learning model to identify matching tracking codes that correspond to the diagnostic terms in the text block; and
returning the matching tracking codes.

22. The method of claim 21, wherein using the at least one trained machine learning model to identify the matching tracking codes that correspond to the diagnostic terms comprises:
submitting the text block to a trained large language model (LLM) with a first prompt for the LLM to return diagnostic term information identifying the diagnostic terms; and
receiving the diagnostic term information from the LLM.

23. The method of claim 22, wherein using the at least one trained machine learning model to identify the matching tracking codes that correspond to the diagnostic terms further comprises:
calculating diagnostic term text embeddings for the identified diagnostic terms; and
comparing the diagnostic term text embeddings to tracking code text embeddings for the tracking codes to identify as candidate tracking codes those of the tracking codes whose respective tracking code text embeddings most closely match the diagnostic term text embeddings.

24. The method of claim 23, wherein using the at least one trained machine learning model to identify the matching tracking codes that correspond to the diagnostic terms further comprises:
sending highest ranked ones of the candidate tracking codes to the LLM along with the text block and a second prompt for the LLM to return the matching tracking codes; and
receiving the matching tracking codes from the LLM.

25. The method of claim 21, wherein using the at least one trained machine learning model to identify the matching codes that correspond to the diagnostic terms comprises:
using an identifier machine learning model to obtain diagnostic term information identifying diagnostic terms within the text block.

26. The method of claim 25, wherein using the at least one trained machine learning model to identify the matching tracking codes that correspond to the diagnostic terms comprises:
using a mapping machine learning model to map the diagnostic terms to corresponding respective tracking codes.

27. The method of claim 21, wherein using the at least one trained machine learning model to identify the matching tracking codes that correspond to the diagnostic terms comprises using a single trained machine learning model to identify the corresponding tracking codes that correspond to the diagnostic terms.

28. The method of claim 21, wherein the text block is a transcript of an audio stream containing spoken words received by a microphone of a data processing system.

29. A data processing system comprising at least one processor and memory coupled to the at least one processor, wherein the memory contains instructions which, when executed by the at least one processor, cause the at least one processor to carry out a method according to claim 21.

30. A computer program product comprising at least one tangible non-transitory computer-readable medium containing instructions which, when executed by at least one processor of a data processing system, cause the data processing system to carry out a method according to claim 21.
